SEQUENCE LISTING (12 sequences)

SEQ ID NO: 1 - protein, 266 aa, Artificial
  Repebody-MD2
ETITVSTPIKQIFPDDAFAETIKANLKKKSVTDAVTQNELNSIDQIIANNSDIKSVQGIQYLPNVRYLALGGNKLHDISALKELTNLTYLILTGNQLQSLPNGVFDKLTNLKELVLVENQLQSLPDGVFDKLTNLTYLNLAHNQLQSLPKGVFDKLTNLTELDLSYNQLQSLPEGVFDKLTQLKDLRLYQNQLKSVPDGVFDRLTSLQYIWLHDNPWDCTCPGIRYLSEWINKHSGVVRNSAGSVAPDSAKCSGSGKPVRSIICPT

SEQ ID NO: 2 - DNA, 4615 bp, Artificial
  pBEL118N
acatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagaccatggccatgaaaataaaaacaggtgcacgcatcctcgcattatccgcattaacgacgatgatgttttccgcctcggctctcgccgaattcgaaaccattaccgtgagcaccccgatcaaacagatttttccggatgacgcgttcgccgaaacgatcaaagcaaacctgaagaaaaagagcgttaccgatgctgtcacgcaaaatgaactgaacagtattgaccagatcattgcgaataactccgatatcaaatcagtgcaaggcattcagtatctgccgaatgttcgttacctggccctgggtggcaacaaactgcatgacatctcggcactgaaagaactgaccaatctgacgtatctgnnkctgnnknnkaaccaactgcagagcctgccgaacggcgtgtttgataaactgacgaacctgaaagaactgnnkctgnnknnkaatcaactgcagtctctgccggatggtgtgttcgacaaactgaccaacctgacgtacctgaatctggctcacaaccaactgcagagtctgccgaaaggcgtgtttgacaaactgaccaatctgacggaactggatctgtcctataaccaactgcagtcactgccggaaggtgttttcgacaaactgacccagctgaaagatctgcgcctgtaccagaatcagctgaaatcggtcccggacggcgtgtttgatcgtctgaccagcctgcagtatatctggctgcatgataacccgtgggattgcacctgtccgggtattcgctacctgtctgaatggatcaataaacacagtggcgttgtccgtaactccgcgggttcagttgccccggattcggcgaaatgctccggcagcggtaaaccggtgcgtagcattatttgcccgacctcgagcaccaccaccaccaccactagggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttagtaaggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgtcaggtggcacttttcggggaaatgtgcgcggaacccctatttgtttatttttctaaatacattcaaatatgtatccgctcatgagacaataaccctgataaatgcttcaataatattgaaaaaagaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc

SEQ ID NO: 3 - DNA, 60 bp, Artificial
  mutagenic primer for module 2
cgagatgtcatgcagtttgttmnnmnncagmnncagmnnacgaacattcggcagatactg

SEQ ID NO: 4 - DNA, 96 bp, Artificial
  mutagenic primer for module 3
tttatcaaacacgccgttcggcaggctctgcagttggttmnnmnncagmnncagatacgtcagattggtcagttctttcagtgccgagatgtcatg

SEQ ID NO: 5 - DNA, 96 bp, Artificial
  mutagenic primer for module 4
tttgtcgaacacaccatccggcagagactgcagttgattmnnmnncagmnncagttctttcaggttcgtcagtttatcaaacacgccgttcggcag

SEQ ID NO: 6 - DNA, 93 bp, Artificial
  mutagenic primer for module 5
cttatcgaagacacctttcggcagactctgcagttggttmnnmnncagmnncagmnncgtcaggttggtcagtttgtcgaacacaccatccgg

SEQ ID NO: 7 - protein, 266 aa, Artificial
  Repebody-D3
ETITVSTPIKQIFPDDAFAETIKANLKKKSVTDAVTQNELNSIDQIIANNSDIKSVQGIQYLPNVRYLALGGNKLHDISALKELTNLTYLVLEPNQLQILPNGVFDKLTNLKELALWLNQLQSLPDGVFDKLTNLTYLNLAHNQLQSLPKGVFDKLTNLTELDLSYNQLQSLPEGVFDKLTQLKDLRLYQNQLKSVPDGVFDRLTSLQYIWLHDNPWDCTCPGIRYLSEWINKHSGVVRNSAGSVAPDSAKCSGSGKPVRSIICPT

SEQ ID NO: 8 - protein, 266 aa, Artificial
  Repebody-D3E8
ETITVSTPIKQIFPDDAFAETIKANLKKKSVTDAVTQNELNSIDQIIANNSDIKSVQGIQYLPNVRYLALGGNKLHDISALKELTNLTYLTLEPNQLQSLPNGVFDKLTNLKELQLWANQLQSLPDGVFDKLTNLTYLNLAFNQLQSLPKGVFDKLTNLTELDLSYNQLQSLPKGVFDKLTQLKDLRLYQNQLKSVPDGVFDRLTSLQYIWLHDNPWDCTCPGIRYLSEWINKHSGVVRNSAGSVAPDSAKCSGSGKPVRSIICPT

SEQ ID NO: 9 - protein, 266 aa, Artificial
  Repebody-D3E8C4 (Y102K, A104T, G106V, G107S)
ETITVSTPIKQIFPDDAFAETIKANLKKKSVTDAVTQNELNSIDQIIANNSDIKSVQGIQYLPNVRKLTLVSNKLHDISALKELTNLTYLTLEPNQLQSLPNGVFDKLTNLKELQLWANQLQSLPDGVFDKLTNLTYLNLAFNQLQSLPKGVFDKLTNLTELDLSYNQLQSLPKGVFDKLTQLKDLRLYQNQLKSVPDGVFDRLTSLQYIWLHDNPWDCTCPGIRYLSEWINKHSGVVRNSAGSVAPDSAKCSGSGKPVRSIICPT

SEQ ID NO: 10 - protein, 266 aa, Artificial
  Repebody-D3E8-KE, D3E8(I82K, R222E)
ETITVSTPIKQIFPDDAFAETIKANLKKKSVTDAVTQNELNSIDQIKANNSDIKSVQGIQYLPNVRYLALGGNKLHDISALKELTNLTYLTLEPNQLQSLPNGVFDKLTNLKELQLWANQLQSLPDGVFDKLTNLTYLNLAFNQLQSLPKGVFDKLTNLTELDLSYNQLQSLPKGVFDKLTQLKDLELYQNQLKSVPDGVFDRLTSLQYIWLHDNPWDCTCPGIRYLSEWINKHSGVVRNSAGSVAPDSAKCSGSGKPVRSIICPT

SEQ ID NO: 11 - protein, 266 aa, Artificial
  Repebody-D3E8(I82K)
ETITVSTPIKQIFPDDAFAETIKANLKKKSVTDAVTQNELNSIDQIKANNSDIKSVQGIQYLPNVRYLALGGNKLHDISALKELTNLTYLTLEPNQLQSLPNGVFDKLTNLKELQLWANQLQSLPDGVFDKLTNLTYLNLAFNQLQSLPKGVFDKLTNLTELDLSYNQLQSLPKGVFDKLTQLKDLRLYQNQLKSVPDGVFDRLTSLQYIWLHDNPWDCTCPGIRYLSEWINKHSGVVRNSAGSVAPDSAKCSGSGKPVRSIICPT

SEQ ID NO: 12 - protein, 39 aa, Artificial
  partial N-terminal fragment of Internalin protein
ETITVSTPIKQIFPDDAFAETIKANLKKKSVTDAVTQNE

(19) United States
(12) Patent Application Publication
(10) Pub. No.: US 2015/0133308 A1
(43) Pub. Date: May 14, 2015

(54) METHOD FOR IMPROVING REPEBODY CONTAINING REPEAT MODULES

(71) Applicants: Korea Advanced Institute of Science and Technology, Daejeon (KR); The Industry & Academic Cooperation in Chungnam National University, Daejeon (KR); Korea Basic Science Institute, Daejeon (KR)

(72) Inventors: Hak-Sung Kim, Daejeon (KR); Joong-Jae Lee, Daejeon (KR); Hyun-Ho Kyeong, Daejeon (KR); Jung-Min Choi, Daejeon (KR); Da-Eun Hwang, Daejeon (KR); Hae-Kap Cheong, Chungcheongbuk-do (KR); Hyun Jung Kim, Chungcheongbuk-do (KR); Eun-Kyeong Jo, Daejeon (KR); Chul-Su Yang, Daejeon (KR); Jae-Min Yuk, Daejeon (KR)

(21) Appl. No.: 14/538,778

(22) Filed: Nov. 11, 2014

(30) Foreign Application Priority Data
Nov. 13, 2013 (KR) 10-2013-0137700

(51) Int. Cl. C12N 15/10; C07K 14/00

(52) U.S. Cl. CPC C12N 15/1058; C07K 14/001; C12N 15/1037; C07K 2318/20; C07K 2319/03
USPC 506/1; 506/18; 506/17; 506/9

(57) ABSTRACT

The present invention relates to a method for improving a repebody protein comprising repeat modules and a nucleotide library encoding a repebody protein library for improving the repebody protein. More particularly, the present invention relates to a method for improving a repebody protein using a module evolution method of sequentially mutating repeat modules constituting the repebody protein, and a nucleotide library encoding a repebody protein library used to improve the protein. According to the module evolution method of the present invention, an improved repebody protein can be screened which has a high binding affinity and accordingly increased specificity and activity, and thus it is easy to express a repebody used as an inhibitor, a therapeutic agent, and an analysis means against a target substance.

### CROSS-REFERENCE TO RELATED APPLICATION

This application claims priority under the provisions of 35 U.S.C. §119 of Korean Patent Application No. 10-2013-0137700 filed Nov. 13, 2013. The disclosure of such Korean priority patent application is hereby incorporated herein by reference in its entirety, for all purposes.

### TECHNICAL FIELD

The present invention relates to a method for improving a repebody protein comprising repeat modules and a nucleotide library encoding a repebody protein library for improving the repebody protein. More particularly, the present invention relates to a method for improving a repebody protein using a module evolution method of sequentially mutating repeat modules constituting the repebody protein, and a nucleotide library encoding a repebody protein library used to improve the protein.

### BACKGROUND ART

Among a variety of substances present in the body, proteins are large molecules that are essential for maintaining the phenomena of life and perform a wide range of biological functions in the body. In order to perform such functions, protein-protein interactions are essential, which can account for all the phenomena of life. If the protein-protein interactions are not properly regulated, homeostasis in the body will be broken, resulting in various diseases. Thus, various methods for artificially regulating the protein-protein interactions to treat diseases have been studied and developed. In fact, various drugs that can be used for therapeutic purposes have been successfully developed.

Antibodies are proteins important for immune responses, and perform biological functions by specific interaction with antigens. This specific binding activity of antibodies makes it possible to achieve high therapeutic effects, unlike conventional small molecular chemical agents. Thus, many research institutes and pharmaceutical companies have actively conducted studies to develop antibodies having binding affinity for well-known therapeutic targets by the use of various screening techniques. Therapeutic antibodies developed for recent decades exhibited high therapeutic effects against various diseases together with low side effects, and thus were significantly successful in the therapeutic drug market and have continuously maintained high growth rates in the therapeutic drug market. Such antibodies have been used in a wide range of applications, including the isolation and purification of biomaterials, and biomedical diagnosis, in addition to therapeutic purposes. Despite such advantages, the development of new therapeutic agents based on antibodies was not so much successful due to problems, including low tissue penetrability resulting from the high molecular weight of antibodies, high production costs resulting from complex production processes, and entry barriers such as existing patents. To overcome such limitations, several research groups have actively conducted studies on new protein backbones for use as a substitute for antibodies. As a result, a protein, named “repebody” was developed. The repebody means a polypeptide prepared by fusion of the N-terminus of internalin protein having a leucine-rich repeat (LRR) structure and VLR based on the structural similarity therebetween so as to have an optimal consensus design. The repebody can be expressed in large amounts in E. coli, and thus can be produced at low costs. Further, it has high physical and chemical stabilities, and thus can be easily modified. In addition, it is a novel protein backbone that has not been studied and developed yet, and this is free from existing patents.

However, in order to improve the therapeutic effect of the repebody as a therapeutic agent, the analysis sensitivity of the repebody as an antibody replacement for analysis, and the efficiency with which the repebody migrates to its target, the affinity of the repebody protein for its target still needs to be increased.

Accordingly, the present inventors have made extensive efforts to increase the binding affinity of the repebody for its target, and as a result, have found that, when mutations in the binding sites of repebody modules, in which protein-protein interactions occur, are sequentially and repeatedly performed using a mutant library for each module, a repebody having a significantly high binding affinity for its target can be screened, thereby completing the present invention.

### DISCLOSURE

### Technical Problem

It is an object of the present invention to provide a repebody protein library comprising two or more repebodies in which each of the repebodies is composed repeat modules, and at least one amino acid in the repeat modules of the repebodies is different between the repeat modules.

Another object of the present invention is to provide a nucleotide library encoding a repebody protein library comprising a plurality of repebodies, each of the repebodies being composed of repeat modules comprising a mutant, and a constructing method thereof.

Still another object of the present invention is to provide a vector library comprising the above-described nucleotide library and a host cell library comprising the above-described vector library.

Yet another object of the present invention is to provide a method for improving a repebody protein comprising repeat modules and having an increased binding affinity for a target.

### Technical Solution

To achieve the above objects, the present invention provides a repebody protein library comprising two or more repebodies having the ability to bind to a target, in which each of the repebodies is composed of repeat modules, each consisting of two or more amino acids, in which each repebody has a concave region serving to recognize a biomolecule and a convex region serving to maintain the structure thereof, the concave region and the convex region being formed by the repeat modules, and in which at least one amino acid in the repeat modules of the repebodies is different between the repeat modules.

The present invention also provides a nucleotide library encoding a repebody protein library comprising two or more repebodies having the ability to bind to a target, in which each of the repebodies is composed of repeat modules, each consisting of two or more amino acids, in which each repebody has a concave region serving to recognize a biomolecule and a convex region serving to maintain the structure thereof, the concave region and the convex region being formed by the repeat modules, and in which at least one amino acid in the repeat modules of the repebodies is different between the repeat modules.

The present invention also provides a vector library comprising the above-described nucleotide library and a host cell library comprising the above-described vector library.

The present invention also provides a method for constructing the above-described nucleotide library, the method comprising the steps of: (a) confirming repeat modules in a template repebody, and determining amino acid residues that are to be used to randomize a selected region of the repeat modules, in view of interaction with a target substance; and (b) constructing a combination of nucleotide sequences encoding one or more of the repeat modules determined in step (a).

The present invention also provides a method for improving a repebody protein, the method comprising the steps of: (a) confirming a concave region of a template repebody, which serves to recognize a biomolecule, and a convex region that serves to maintain the structure of the template repebody, and determining amino acid residues to be randomized, in view of interaction with a target substance; (b) constructing a protein library including repeat modules having the randomized amino acid residues; (c) selecting, from the protein library, a repebody having an increased binding affinity for the target substance; (d) confirming a mutated region in the repebody selected in step (c), and determining amino acid residues to be randomized in modules adjacent to the mutated region; (e) constructing a protein library including repeat modules having the randomized amino acid residues determined in step (d); (f) selecting, from the protein library of step (e), a repebody having an increased binding affinity for the target substance; and (g) repeating steps (d) to (f) n times.

According to the module evolution method of the present invention, an improved repebody protein can be screened which has a high binding affinity and accordingly increased specificity and activity, and thus it is easy to express a repebody used as an inhibitor, a therapeutic agent, and an analysis means against a target substance.

### BRIEF DESCRIPTION OF THE DRAWINGS

FIG. 1 is a schematic view showing the overall protein structure of repebody.

FIG. 2 is a graphic diagram showing the position of a repebody selected in the present invention and dissociation constant graphs (a: repebody; B:r-D3, C:r-D3E8; D:r-D3E8C4).

FIG. 3 is an overall schematic view showing an overlap polymerase chain reaction (PCR) performed based on modules. Each yellow portion is a variable repeat module, and a total of four variable repeat modules are located on a polypeptide. The linear bars indicated by a red color are primers used in the experiment, and a green portion in the primers indicates the sequence of a concave area including an NNK consensus codon.

FIG. 4 is a schematic view showing the crystallized structure of a complex of repebody and interleukin-6.

FIG. 5 is a graphic diagram showing the results obtained by treating a medium of non-small-cell lung cancer cells with peptides (D3, D3E8, D3E8C4, D3E8 (182K), and D3E8-KE) and measuring changes in STATS activity and interleukin-6 production.

FIG. 6 is a graphic diagram showing the results obtained by treating non-small-cell lung cancer cells with repebodies (D3, D3E8, D3E8C4, D3E8 (182K), and D3E8-KE), and then subjecting the cells to an MTT assay to measure cell viability.

FIG. 7 is a graphic diagram showing the results obtained by intraperitoneally injecting a polypeptide (D3E8-KE) and a control (PBS) four times at 3-day intervals for 10 days into xenograft mice injected with non-small-cell lung cancer cells, and then measuring a change in the tumor volume.

FIG. 8 is a graphic diagram showing the results obtained by intraperitoneally injecting a repebody (D3E8-KE) and a control (PBS) five times at 3-day intervals for 15 days into xenograft mice injected with non-small-cell lung cancer cells, and then measuring a change in the tumor volume.

### DESCRIPTION

In one aspect, the present invention is directed to a repebody protein library comprising two or more repebodies having the ability to bind to a target substance, in which each of the repebodies is composed of repeat modules, each consisting of two or more amino acids, in which each repebody has a concave region serving to recognize a biomolecule and a convex region serving to maintain the structure thereof, the concave region and the convex region being formed by the repeat modules, and in which at least one amino acid in the repeat modules of the repebodies is different between the repeat modules, a nucleotide library encoding the repebody protein library, and a method for constructing the nucleotide library.

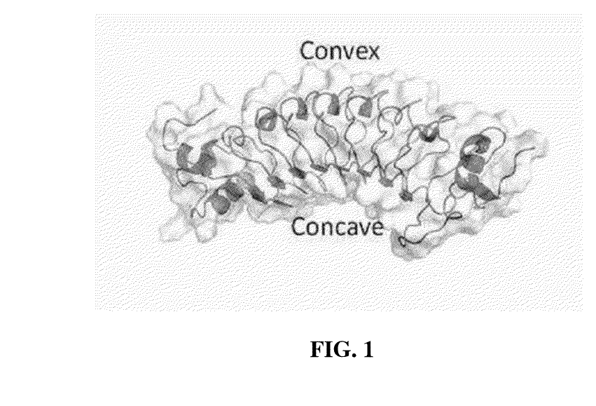

The repebody consists of continuously connected repeat units having conserved leucine sequence, similar to LRR proteins present in the natural world and has a modularity maintaining the entire protein structure and structural characteristic of a concave region and a convex region differentiated by curvature of the entire structure (FIG. 1). FIG. 1 is a schematic diagram showing an entire protein structure of repebody, divided into a concave region recognizing biomolecule and a convex region which is important in maintaining the structure. A hypervariable region like a complementarity determining region (CDR) was positioned in the concave region to mediate a protein-protein interaction. In addition, the convex region is important to maintain the entire structure of LRR based on the well conserved sequence. The protein structure of the repebody was analyzed and a random library was designed by the following scheme.

In addition, the library may be formed of phagemid including the polynucleotide. In the present invention, the term “phagemid” means a circular polynucleotide molecule derived from a phage which is a virus having E. coli as a host and includes sequences of proteins and surface-proteins required for propagation and proliferation. A recombinant phagemid may be produced using gene recombinant technology well known in the art, and site-specific DNA cleavage and connection may be performed by an enzyme, generally known in the art, and the like. The phagemid may include a signal sequence or leader sequence for secretion in addition to expression regulating factors such as a promoter, an operator, an initiation codon, a termination codon, an enhancer and may be mainly used in a method for labeling the protein on a surface of the phage by fusing a desired protein with a surface protein of the phage. The promoter of the phagemid is mostly inducible and may include a selective marker for selecting a host cell.

For an object of the present invention, the phagemid may be a polynucleotide of SEQ ID NO: 2, including MalEss, DsbAss or PelBss which is a signal sequence or a leader sequence for expressing and secreting the polynucleotide which encodes the polypeptide constructing the library, and including a histidine-tag for confirming expression of a recombinant protein on a surface of the phage, and a polynucleotide which encodes gp3 domain which is a kind of a surface protein of M13 phage for expression on the surface of the phage, but the present invention is not particularly limited thereto.

In the present invention, the repeat modules may be 2-30 in number, the repeat modules may be directly linked to each other, or the repeat modules may be linked together by a (poly)peptide linker, but are not limited thereto.

In the present invention, a protein, from which the N-terminus or C-terminus of the repeat modules is derived, may be different between the repeat modules, and the N-terminus of the repeat modules may be the N-terminus of an LRR (leucine rich repeat) protein that is of microbial origin and has an alpha helical capping motif.

In the present invention, the N-terminus of the protein may be a fusion polypeptide, which has an amino acid sequence of SEQ ID NO: 12 and is composed of the following repeat module pattern:

LxxLxxLxLxxN

wherein L is alanine, glycine, phenylalanine, tyrosine, leucine, isoleucine, valine or tryptophan, N is asparagine, glutamine, serine, cysteine or threonine, and x is a hydrophilic amino acid.

In the present invention, the protein may be selected from the group consisting of internalin protein A, internalin protein B, internalin protein C, internalin protein H and internalin protein J, and the C-terminus of the repeat modules may be of microbial origin and may be the C-terminus of VLR (Variable Lymphocyte Receptor) protein.

In addition, the repeat modules may be modified repeat modules of VLR (Variable Lymphocyte Receptor) protein, and the modified repeat modules of the VLR protein may comprise the following repeat module pattern:

LxxLxxLxLxxN

wherein L is alanine, glycine, phenylalanine, tyrosine, leucine, isoleucine, valine or tryptophan, N is asparagine, glutamine, serine, cysteine or threonine, and x is any amino acid.

In another aspect, the present invention is directed to a method for improving a repebody protein, the method comprising the steps of: (a) confirming a concave region of a template repebody, which serves to recognize a biomolecule, and a convex region that serves to maintain the structure of the template repebody, and determining amino acid residues to be randomized in view of interaction with a target; (b) constructing a protein library including repeat modules having the randomized amino acid residues; (c) selecting, from the protein library, a repebody having an increased binding affinity for the target substance; (d) confirming a mutated region in the repebody selected in step (c), and determining amino acid residues to be randomized in modules adjacent to the mutated region; (e) constructing a protein library including repeat modules having the randomized amino acid residues determined in step (d); (f) selecting, from the protein library of step (e), a repebody having an increased binding affinity for the target substance; and (g) repeating steps (d) to (f) n times.

In the present invention, the library in step (b) may be constructed by a phage display method, and the phage display method may be a method of phage-displaying a product obtained from a template repebody-encoding DNA by an overlapping PCR method.

In the present invention, the method may further comprise a step of performing additional mutation in a region of the selected repebody, which is to bind to the target substance, based on the structural analysis of a complex consisting of the selected repebody bound to the target substance.

The present invention is based on a module type manipulation method in which a protein-protein binding site can be more efficiently manipulated. An approach to a module level for increasing the binding affinity of a protein complex is a method that is more effective and simple than a traditional method.

In the present invention, a protein-protein binding site was optimized stepwise by a module-by-module (module-by-module method to develop a method capable of increasing the binding affinity up to the picomolar unit.

The method according to the present invention is useful to regulate the protein-protein binding site, particularly is useful to extend and optimize the functional surface of a protein structurally composed of homologous motifs.

Recently, a study has been conducted on an array in which multiple LRR modules are re-arranged and combined to form a receptor for an antigen in the immune system of jawless vertebrates (Pancer, Z. et al. Nature 430:174, 2004; Alder, M. N. et al., Science 310:1970, 2005).

The module evolution method according to the present invention includes the above-described combination process including a repeat library construction route and a module-based selection.

In an example of the present invention, an anti-human IL-6 repebody was constructed in which the binding affinity is increased 120-fold by being increased up to 397 pM through the use of the module evolution method using r-D3(K_{D}=47 nM) which is a repebody against human IL-6(hIL-6) as a starting material.

The module evolution method according to the present invention enables random residues to be more easily confirmed even without any information on interaction sites than in the conventional method, and various libraries can be effectively constructed with motifs confirmed through a combination of modules.

In another example of the present invention, it was found that multi-electorstatic and hydrophobic interactions play a key role in the formation of an r-D3E8/hIL6 complex as a conjugate of r-D3E8 repebody produced by the module evolution method and hIL-6. In particular, it was found that the positive charge residues (R24, K27 and R30) of an α-helical site of hIL-6 play a key role in the increase of the binding affinity thereof. In addition, it was found that the hydrophobic residues of modules 4 and 5 of r-D3E8 cause the hydrophobic interactions with hIL-6, which plays a key role in the increase of the binding affinity, and that the binding affinity for hIL-6 was optimized to 63 pM and thus the efficiency of a module design was demonstrated. The binding site between two proteins is formed in a modular fashion indicating a low interaction between modules, and thus the module-by-module approach is very efficient in the manipulation of the protein-protein binding site.

In order to develop a protein complex acting as an hIL-6-related disease therapeutic agent, it is required that the protein complex should be capable of efficiently suppressing the formation of a hexameric complex through the binding of hIL-6 to IL-6Rα or gp130.

As a result of comparison of the crystal structure between the r-D3E8/hIL6 complex (PDB (protein data bank) ID 4J4L) and the hexameric complex (PDB ID 1P9M), it was found that there is a match between the binding mode of r-D3E8 and IL-6Rα/gp130 as well as many antigen recognition site (epitope) residues in both complexes. That is, it was found that Arg30 and Arg182 of helix A and helix D of hIL-6 are important in the binding between hIL-6 and IL-6Rα.

In another example of the present invention, it was found that a strong inhibitory effect of the repebody on hIL-6 in an hIL-6-related signal process results from the effective suppression of the binding of IL-6Rα and gp130 to hIL-6. This suggests that the repebody production method of the present invention can also be used in competitive immune analysis employing an antibody for hIL-6, of which epitope is known.

In another example of the present invention, it was found that the repebody r-D3E8-KE manufactured by the inventive method exhibits a high anti-cancer activity through the suppression of proliferation of tumor cells when being administered to mice whose tumor cells are being proliferated. This high anti-cancer activity is caused by a high binding affinity (63 pM) of r-D3E8-KE for hIL-6, which demonstrates the efficiency of manipulation of the protein-protein binding site by the module evolution method according to the present invention.

As used herein, the term “interleukin-6 (IL-6)” refers to a glycoprotein having a molecular weight of 210,000 and isolated with B cell stimulatory factor-2 (BSF-2) that induces the final differentiation of B cells into antibody-producing cells. It is a kind of cytokine that is produced in various cells, including T lymphocytes, B lymphocytes, macrophages, fibroblasts and the like, and is involved in immune responses, the proliferation and differentiation of cells of the hematopoietic system and the nervous system, acute reactions, the growth and differentiation of various cells, etc.

In the present invention, the term “repebody” is a polypeptide optimized by consensus design through fusion of the N-terminal of the internalin B having the LRR protein structure and the VLR based on the structural similarity. The repebody protein may include all fusion LRR family proteins obtained by using all proteins included in the LRR family having the repeat module to improve the solubility expression and biophysical properties of protein of all proteins by the above-described method.

In the present invention, the term “internalin B protein” is a kind of the LRR family protein expressed in a Listeria strain, and it is known that the internalin protein has an N-terminal structure different from that of the LRR family proteins in which a hydrophobic core are uniformly distributed through the entire molecule to thereby be stably expressed in microorganisms. It is considered that since the N-terminal of the internalin B protein which is the most important in folding a repeat module is derived from a microorganism and has a stable shape including an alpha helical, such that the internalin protein is stably expressed in microorganisms.

In the present invention, the term “N-terminal of an (or the) internalin B protein” of the present invention means an N-terminal of the internalin B protein required for soluble expression and folding of the protein, and means a repeat module of the alpha helical capping motif and the internalin B protein. The N-terminal of the internalin B protein may limitlessly include any N-terminal of the internalin B protein required for soluble expression and folding of the protein, and as an example thereof, an alpha helical capping motif “ETITVSTPIKQIFPDDAFAETIKANLKKKSVTDAVTQNE” and the repeat module may be included. Preferably, the repeat module pattern may be “LxxLxxLxLxxN”. In the repeat module pattern, L means alanine, glycine, phenylalanine, tyrosine, leucine, isoleucine, valine, or tryptophan; N means asparagine, glutamine, serine, cysteine or threonine; and x means any amino acid. In addition, the N-terminal of the internalin B protein of the present invention may be used without limitations as long as the N-terminal has a high structural similarity depending on a kind of the LRR family protein that can be fused, and the most stable amino acid may be selected by calculation of a binding energy, and the like, and the amino acid of the module corresponding thereto may be mutated.

In the present invention, the term “VLR (Variable Lymphocyte Receptor) is a kind of the LRR family protein that is expressed and performs an immune function in hagfishes and lampreys, and is coming into the spotlight as a backbone capable of binding to various antigenic substances. A polypeptide in which the N-terminal of the internalin B protein and the VLR protein are fused is relatively increased in solubility and expression amount as compared to a VLR Protein that is not fused with the internalin B protein, and thus can be usefully used in the preparation of a novel protein therapeutic agent based on the increase of solubility and expression amount. Such an increase of the expression amount suggests that when the polypeptide of the present invention is used, the economic efficiency can be significantly improved.

In the present invention, the term “modified repeat module of VLR protein” means that among TV3 proteins known as inherently interacting with MD-2, an important TV3 amino acid known as binding to MD-2 is grafted onto a repeat module of a relevant VLR protein in a module in which leucine consisting of 24 amino acids is repeated at a certain position.

The LRR repeat module has “LxxLxxLxLxxN” as a conservation pattern, wherein L means hydrophobic amino acids such as alanine, glycine, phenylalanine, tyrosine, leucine, isoleucine, valine, and tryptophan; N means asparagine, glutamine, serine, cysteine or threonine and x means any amino acid. The LRR family protein of the present invention may include all mutants having the sequence which is already known or found by newly induced mRNA or cDNA, as well as the sequence which is not known in the natural world through consensus design, and the like, and having a frame of the repeat module.

In the present invention, the term “Leucine rich repeat (LRR) family protein” means a protein formed by combination of modules in which leucine is repeated at a certain position, (i) it has one or more LRR repeat modules, (ii) the LRR repeat module consists of 20 to 30 amino acids, (iii) the LRR repeat module has “LxxLxxLxLxxN” as a conservation pattern, wherein L means hydrophobic aminoacids such as alanine, glycine, phenylalanine, tyrosine, leucine, isoleucine, valine, and tryptophan; N means asparagine, glutamine, serine, cysteine or threonine and x means any amino acid, and (iv) the LRR family protein means a protein having a three dimensional structure like horseshoe. The LRR family protein of the present invention may include all mutants having the sequence which is already known or found by newly induced mRNA or cDNA, as well as the sequence which is not known in the natural world through consensus design, and the like, and having a frame of the repeat module.

In still another aspect, the present invention is directed to a repebody protein library encoded by the above-described nucleotide library and comprising two or more repebodies having the ability to bind to a target substance, in which each of the repebodies is composed of repeat modules, each consisting of two or more amino acids, in which each repebody has a concave region serving to recognize a biomolecule and a convex region serving to maintain the structure thereof, the concave region and the convex region being formed by the repeat modules, and in which at least one amino acid in the repeat modules of the repebodies is different between the repeat modules.

In the present invention, the term “mutation” or “modification” may include all substitution, deletion, or insertion of amino acid residues, but may preferably include substitution of the existing amino acid residue with the other amino acid residue.

In yet another aspect, the present invention is directed to a vector library comprising the above-described nucleotide library and a host cell library comprising the above-described vector library.

In the present invention, the term “vector” may be a DNA product containing base sequence of polynucleotide encoding a target protein operably connected to an appropriate regulation sequence so as to express the target protein in a suitable host cell. The regulation sequence may include a promoter capable of initiating transcription, an any operator sequence for regulating transcription, a sequence encoding an appropriate mRNA ribosome binding site, and a sequence regulating termination of transcription and decoding and may be variously produced depending on a purpose. The promoter of the vector may be constitutive or inducible. The vector may be transfected into a suitable host and then may be replicated or may perform functions regardless of the host genome, and may be integrated into a genome itself.

The vector used in the present invention is not particularly limited as long as it is replicated in host cells, and may be any vector known in the art. Examples of the generally used vector may include plasmid, phagemid, cosmid, virus, and bacteriophage in a natural state or a recombinant state. For example, as the phage vector or the cosmid vector, pWE15, M13, λMBL3, λMBL4, λIXII, λASHII, λAPII, λt10, λt11, Charon4A, and Charon21A may be used, and as the plastmid vector, pBR-based, pUC-based, pBluescriptII-based, pGEM-based, pTZ-based, pCL-based and pET-based, may be used. The vector usable in the present invention is not particularly limited but may be any known expression vector. Preferably, pACYC177, pACYC184, pCL, pECCG117, pUC19, pBR322, pMW118, pCC1BAC vectors, and the like, may be used. Most preferably, pACYC177, pCL and pCC1BAC vectors may be used.

In the present invention, the term “recombinant microorganism” means a transfected cell in which a vector having polynucleotide encoding one or more target proteins is introduced into a host cell, or polynucleotide encoding one or more target proteins is introduced into a microorganism, such that the polynucleotide is integrated into the chromosome to express the target protein, and may include all cells of eukaryotic cells, prokaryotic cells, and the like. Examples thereof may include bacteria cells such as E. coli, streptomyces, salmonella typhimurium, and the like; yeast cells; fungus cells such as pichiapastoris, and the like; insect cells such as drosophila, spodoptera Sf9 cell, and the like; animal cells such as CHO, COS, NSO, 293, bow melanoma cell, and the like, but the present invention is not particularly limited thereto.

In the present invention, the term “transfection” means that a vector containing polynucleotide encoding a target protein is introduced into a host cell, or a polynucleotide encoding a target protein is integratedly completed into chromosome of the host cell, such that protein encoded by the polynucleotide is capable of being expressed in the host cell. The transfected polynucleotide may be any one regardless of the position as long as the polynucleotide is capable of being expressed in the host cell, regardless of the matter that the polynucleotide is inserted and positioned into chromosome of the host cell or positioned on an outer portion of the chromosome. In addition, the polynucleotide includes DNA and RNA encoding the target protein. The polynucleotide may be inserted with any type as long as the polynucleotide is capable of being introduced into the host cell to be expressed. For example, the polynucleotide may be introduced into the host cell as an expression cassette which is a gene structure, including all factors required for self expression. The expression cassette may include a promoter, transcription termination signal, ribosome binding site and translation termination signal which may be operably connected to the polynucleotide. The expression cassette may be an expression vector performing self-replication. In addition, the polynucleotide may be introduced into the host cell as itself to be operably connected to the sequence required for expression in the host cell.

In the method, the culturing of the transformant may be preferably performed by a batch culture method, a continuous culture method, a fed-batch culture, and the like, known in the art, but the present invention not particularly limited thereto, wherein under the culture condition, pH may be appropriately adjusted (pH 5 to 9, preferably pH 6 to 8, most preferably pH 6.8) by using a basic compound (for example: sodium hydroxide, potassium hydroxide or ammonia) or an acidic compound (for example, phosphoric acid or sulfuric acid), and an aerobic condition may be maintained by introducing oxygen, or an oxygen-containing gas mixture into the culture, and the culture may be performed at 20 to 45° C., preferably, 25 to 40° C. for about 10 to 160 hours. The repebody produced by the culture may be secreted in the medium or remained in the cell.

In addition, in the culture medium used, as carbon source, sugar and carbohydrate (for example, glucose, sucrose, lactose, fructose, maltose, molasse, starch and cellulose), oil and fat (for example, soybean oil, sunflower seed oil, peanut oil and coconut oil), fatty acid (for example, palmitic acid, stearic acid and linoleic acid), alcohol (for example, glycerol and ethanol) and organic acid (for example, acetic acid), and the like, may be used individually or by mixing; as nitrogen source, nitrogen-containing organic compound (for example, peptone, yeast extract, gravy, malt extract, corn steep liquor, soybean meal powder and urea), or inorganic compound (for example, ammonium sulfate, ammonium chloride, ammonium phosphate, ammonium carbonate and ammonium nitrate) and the like, may be used individually or by mixing; as phosphate source, potassium dihydrogen phosphate, dipotassium hydrogen phosphate, sodium-containing salt corresponding thereof, and the like, may be used individually or by mixing; or essential growth-promoting materials such as other metal salts (for example, magnesium sulfate or iron sulfate), amino acids and vitamins may be included.

In the recovering of the repebody produced in the culturing of the present invention, the desired repebody may be recovered from a culture fluid by appropriate culture methods such as a batch culture method, a continuous culture method, a fed-batch culture, and the like, known in the art.

### EXAMPLES

Hereinafter, the present invention will be described in further detail with reference to examples. It will be obvious to a person having ordinary skill in the art that these examples are illustrative purposes only and are not to be construed to limit the scope of the present invention. In addition, it will be apparent to those skilled in that art that various modifications and variations can be made without departing from the technical scope of the present invention based on this illustration.

### Example 1

### Design of Phagemid for Selection of Random Repebody Library

A protein backbone named repebody was used as a component of the present invention. The backbone is a water soluble polypeptide in which an LRR portion containing an N-terminal of an internalin B protein and a C-terminal of VLR protein is fused, and has an amino acid sequence the same as SEQ ID NO: 1.

### Example 1-1

### Expression of Repebody Using Signal Sequence in Periplasm

In order to confirm whether or not the repebody is applicable to a phage display, it is required to confirm periplasmic expression of E. coli to be used as a host, and whether or not protein is well expressed onto a surface particle of a phage. To this end, two recombinant vectors were produced by inserting MalE and DsbA signal sequences which are signal polypeptides differentiated from each other right into the back side of an initiation codon, using pMAL-c2x (NEB, USA) vector. Then, DNA in which the repebody and a histidine-tag are fused was inserted between the signal sequence and termination codon to complete a final vector. Two completed vector was introduced into E. coli XL1-blue strain to produce a transformant, the transformant was cultured until absorbance (OD₆₀₀) reached 0.5 and then 0.1 mM IPTG (Isopropyl β-D-1-thiogalactopyranoside) was treated to induce expression of the protein, followed by culturing at 30° C. for 16 hours again. After the culturing was completed, the strain was obtained by centrifugation and treated by ultrasonic wave to obtain a water soluble protein fraction.

The obtained water soluble protein fraction was applied to a Ni-NTA (Nickel-nitrilotriacetic acid) resin to be purified, and an expression amount of the produced repebody in periplasm was confirmed by SDS-PAGE analysis and it was confirmed that the MalE signal sequence had a periplasmic expression amount slightly higher than that of the DsbA signal sequence.

### Example 1-2

### Construction of Phagemid for Repebody Expression on Surface of Phage

A phagemid was designed based on the MalE signal sequence finally determined in Example 1-1 above. With pTV118N (Takara, Japan) as a basic frame, the MalE signal sequence was inserted right into the back side of the initiation codon and DNA in which the repebody and a histidine-tag are fused was added to thereby construct a phagemid. In addition, gp3 which is capable of labeling a relatively large protein among several phage surface proteins was used, C-terminal was positioned at the back of an amber codon, and two continuous terminal codons were finally inserted thereto, thereby completing the phagemid named pBEL118M (SEQ ID NO: 2). The phagemid was introduced into XL1-Blue to produce a transformant, and the produced transformant was cultured by the same method as Example 1-1 above except for treatment with 0.5 mM IPTG, followed by centrifugation to obtain a culture fluid.

The culture fluid was applied to Polyethylene glycol precipitation method to purify the phage.

The phage was analyzed by Western Blot, and as a result thereof, it was confirmed that the repebody was expressed on a surface of the phage.

### Example 2

### Manipulation of Protein Binding Site by Module Evolution Method

### Example 2-1

### Construction of Primary Library

A hypervariable region like a complementarity determining region (CDR) of an antibody was positioned in a concave region of the repebody to mediate a protein-protein interaction. In addition, the convex region is important to maintain the entire structure of LRR based on the well conserved sequence. The protein structure of the repebody was analyzed and a random library was constructed by the following scheme.

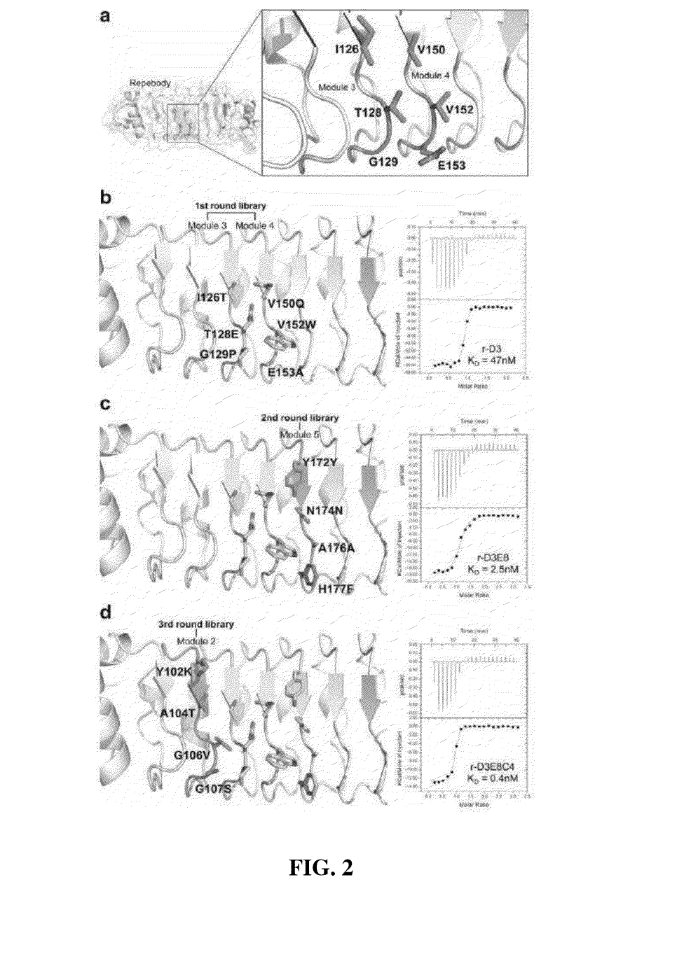

Specifically, a library was constructed to induce random mutations at three hypervariable sites (positions 8, 10 and 11) positioned in modules 3 and 4 of the concave region of the repebody (FIG. 2(a)).

Then, the selected amino acid was substituted with NNK degenerate codon and configured so that base sequences of the other convex region include silent mutation, thereby synthesizing a mutagenic primer for constructing a library.

Module 2 mutagenic primer:
(SEQ ID NO: 3)
CGA GAT GTC ATG CAG TTT GTT MNN MNN CAG MNN CAG
MNN ACG AAC ATT CGG CAG ATA CTG
Module 3 mutagenic primer:
(SEQ ID NO: 4)
TTT ATC AAA CAC GCC GTT CGG CAG GCT CTG CAG TTG
GTT MNN MNN CAG MNNCAG ATA CGT CAG ATT GGT CAG
TTC TTT CAG TGC CGA GAT GTC ATG
Module 4 mutagenic primer:
(SEQ ID NO: 5)
TTT GTC GAA CAC ACC ATC CGG CAG AGA CTG CAG TTG
ATT MNN MNN CAG MNN CAG TTC TTT CAG GTT CGT CAG
TTT ATC AAA CAC GCC GTT CGG CAG
Module 5 mutagenic primer:
(SEQ ID NO: 6)
CTT ATC GAA GAC ACC TTT CGG CAG ACT CTG CAG TTG
GTT MNN MNN CAG MNN CAG MNN CGT CAG GTT GGT CAG
TTT GTC GAA CAC ACC ATC CGG

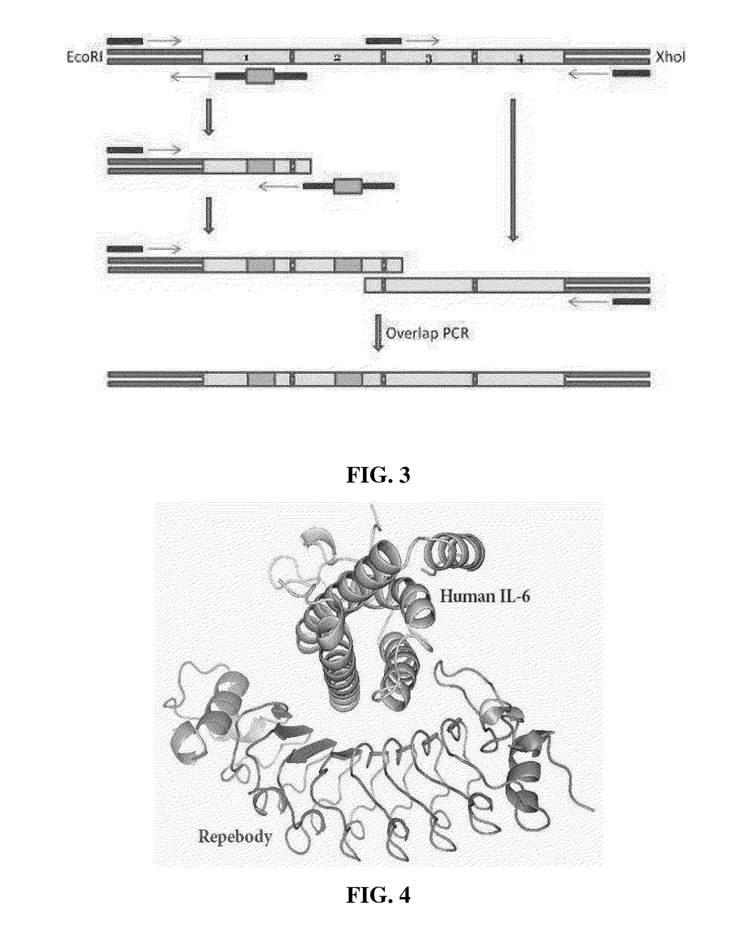

Next, overlap PCR was performed on two modules using the primers to obtain a library DNA (FIG. 3) and the library DNA was inserted into the phagemid pBEL118M to secure a final library phagemid. FIG. 3 is a schematic diagram showing the entire overlap PCR performed based on a module. Each yellow part indicates a variable repeat module and a total of four variable repeat modules are positioned on a polypeptide. A red linear rod indicates a primer used in experiments and a green part of the primer indicates sequence of a concave region containing NNK degenerate codon.

The secured library was introduced into E. coli XL1-Blue by electroporation to obtain a transformant, such that a library having a synthetic diversity with a level of 1.8×10⁸ was constructed.

### Example 2-2

### Selection of Protein Specifically Bound to IL-6 Using Phage Display

The library constructed in Example 2 was cultured by the method of Example 1-1 above and the phage in which the repebody was expressed on a surface thereof was selected by the method of Example 1-2 and purified. In order to select a candidate capable of binding IL-6, 100 ug/ml of IL-6 was coated on an immuno tube at 4° C. for 12 hours or more. The coated tube was washed three times with PBS (Phosphate buffered saline), followed by blocking with a PBS solution (TPBS) containing 1% bovine serum albumin (BSA) and 0.1% Tween 20 at 4° C. for 2 hours. After the blocking, 10¹² cfu (Colony forming unit)/ml of the purified phage was added to the coated tube and was reacted at room temperature for 2 hours. After the reaction has been completed, the reactant was washed with a PBS solution (TPBS) containing 0.1% Tween 20 total five times for 2 minutes and then washed again with PBC twice. Finally, 1 ml 0.2M Gly-HCl (pH2.2) was added to the immune tube, and the reactant was treated with 1 ml 0.2M Gly-HCl (pH2.2) at room temperature for 12 minutes to elute the phage having a candidate capable of binding to IL-6, expressed on the surface thereof in the tube. The reactant after the elution was neutralized with 60 ul of 1.0M Tris-HCl (pH9.1) and 10 ml XL1-Blue (OD₆₀₀=0.5) which is a host cell was inserted thereinto, followed by plating on a 2xYT plate. A bio-panning process through a series of process as described above was performed total of three times. As a result, a phenomenon that the phage specifically bound to IL-6 through each panning process is enriched was observed. The result means that the library phage bound to IL-6 is specifically increased.

### Example 2-3

### Confirmation of Whether the Selected Repebody Binds Specifically to IL-6, and Sequencing of the Selected Repebody

The phages selected by the method of Example 2-2 were subjected to Enzyme-linked immunosorbent assay (ELISA) using a 96-well plate coated with IL-6 and BSA, thereby selecting 84 repebody candidates in which the absorbance (OD450) of IL-6 was at least 10 times higher than that of BSA. The amino acid sequence of each of the candidates was analyzed, and then WebLogo was performed to determine the consensus sequence. As a result, it was shown that residues having a high mutation frequency were present in the amino acid sequences of proteins that did bind specifically to IL-6 expressed in the selected phages.

Specifically, it was shown that the amino acid isoleucine at position 91 was substituted with tryptophan, valine or threonine, the amino acid threonine at position 93 was substituted with arginine or glutamic acid, the amino acid glycine at position 94 was substituted with alanine, serine or proline, the amino acid valine at position 115 was substituted with valine (silent mutation), serine, alanine or asparagine, the amino acid valine at position 117 was substituted with lysine or tryptophan, and the amino acid glutamic acid at position 118 was substituted with arginine, lysine or leucine. Such results suggest that residues playing an important role in binding to IL-6 are present.

### Example 2-4

### Analysis of Characteristics of Repebody that Binds Specifically to Interleukin-6

Among the repebody candidates obtained based on the results of the ELISA performed in Example 3-2, the dissociation constants of the candidates that showed the highest absorbance for IL-6 were measured. Specifically, the dissociation constants of the repebody candidates for IL-6 at room temperature were measured by isothermal titration calorimetry (ITC) using the repebody dissolved in PBS at 0.2 mM (6 mg/ml), and IL-6 dissolved in PBS at 0.02 mM (0.5 mg/ml) (see Table. 1).

TABLE 1

| | Module 3 | | | Module 4 | | | |
|---|---|---|---|---|---|---|---|
| Clone | 126 | 128 | 129 | 150 | 152 | 153 | K_{D} |
| WT | I | T | G | V | V | E | (nM) |
| r-B10 | V | D | P | S | W | T | |
| r-H8 | P | D | G | V | T | S | |
| r-F7 | V | D | P | S | W | T | |
| r-A1 | T | E | P | N | W | L | |
| r-D10 | T | E | V | Q | W | I | |
| r-D3 | T | E | P | Q | W | A | 47 |
| r-D7 | V | E | P | A | W | I | |
| r-C5 | T | E | P | S | W | T | |
| r-B4 | V | E | P | S | W | L | |
| r-E11 | V | E | S | S | W | P | |
| r-E12 | V | E | P | A | W | S | |
| r-A3 | V | E | P | A | W | T | |
| r-A2 | T | E | P | S | W | L | |
| r-C4 | T | E | P | S | W | T | |
| r-F1 | T | E | P | S | W | F | |
| r-C2 | T | E | P | N | W | P | |
| r-E8 | T | E | P | N | W | P | |
| r-C7 | T | E | P | N | W | P | |
| r-C8 | V | E | P | A | W | L | 90 |
| r-A5 | V | E | P | S | W | M | |
| r-B1 | V | E | P | N | W | L | |
| r-B2 | V | E | P | N | W | L | |
| r-D1 | V | E | P | A | W | L | |
| r-E3 | V | E | P | A | W | L | |
| r-B3 | V | E | P | A | W | I | 49 |
| r-B7 | T | E | P | S | W | L | |
| r-C9 | V | E | P | N | W | S | |
| r-C1 | V | E | P | S | W | P | |
| r-D11 | V | E | P | S | W | S | |
| r-F10 | V | E | S | S | W | P | |
| r-G4 | V | E | P | A | W | L | |
| r-F11 | T | E | P | S | W | M | 117 |
| r-G8 | V | E | P | A | W | L | |
| r-G3 | R | E | W | H | Y | E | |
| r-H1 | V | E | P | N | W | L | |
| r-H10 | V | E | P | S | W | L | |
| r-H2 | V | E | P | A | W | L | |
| r-A7 | T | E | P | S | W | V | |
| r-F8 | V | E | P | N | W | L | |
| r-A9 | V | E | P | N | W | L | |
| r-F4 | V | E | P | N | W | L | |
| r-F3 | T | E | P | Q | W | A | |
| r-H5 | V | E | P | A | W | L | |
| r-G5 | D | I | R | G | N | T | |
| r-H3 | G | K | S | F | D | L | |
| r-A4 | D | L | D | E | M | F | |
| r-F9 | R | L | A | Y | S | P | |
| r-G12 | G | P | T | R | V | N | |
| r-B9 | T | Q | P | N | A | G | |
| r-C11 | W | R | S | V | K | R | |
| r-E10 | W | R | S | V | K | R | |
| r-D12 | W | R | S | V | K | R | |
| r-D2 | W | R | S | V | K | R | |
| r-A12 | W | R | A | V | K | R | |
| r-E5 | W | R | A | V | K | R | |
| r-E1 | W | R | A | V | K | R | |
| r-E4 | W | R | A | V | K | R | |
| r-A6 | W | R | A | V | K | R | |
| r-B6 | W | R | A | V | K | R | |
| r-B11 | W | R | A | V | K | R | |
| r-B5 | W | R | A | V | K | R | |
| r-C3 | W | R | A | V | K | R | |
| r-E6 | W | R | A | V | K | R | |
| r-B8 | W | R | A | V | K | R | |
| r-D6 | W | R | A | V | K | R | |
| r-D5 | W | R | G | V | K | R | |
| r-D8 | W | R | A | V | K | K | |
| r-C10 | W | R | A | V | K | R | |
| r-E9 | W | R | S | V | K | R | |
| r-A10 | W | R | A | V | K | R | |
| r-E2 | W | R | A | V | K | K | |
| r-G9 | W | R | A | V | K | R | |
| r-H7 | T | R | R | T | K | A | |
| r-G2 | W | R | A | V | K | K | |
| r-F12 | W | R | A | V | K | R | |
| r-F5 | W | R | A | V | K | R | |
| r-G1 | T | V | I | S | N | R | |
| r-C6 | V | W | P | D | V | V | |
| r-H9 | S | Y | S | P | L | A | |
| r-G11 | W | Y | P | P | P | R | |
| r-F2 | D | Y | V | V | V | E | |
| r-G6 | D | Y | V | V | V | E | |

As can be seen in the table, among the four repebody candidates, the dissociation constant (K_{D}) of repebody-D3 for IL-6 is 47 nM, the dissociation constant (K_{D}) of repebody-B3 for IL-6 is 48 nM, the dissociation constant (K_{D}) of repebody-C3 for IL-6 is 90 nM, and the dissociation constant (K_{D}) of repebody-F11 for IL-6 is 117 nM. Thus, it could be found that among the four candidates, repebody-D3 can most effectively bound to IL-6.

### Example 2-5

### Construction of Secondary Libraries for Module Evolution

For the purpose of the additional improvement of repebody r-D3 selected in Example 2-4, a secondary library was constructed by the module-by-module method. The construction of the secondary library was performed in the same manner as that in Example 2-1, and four positions (positions 6, 8, 10 and 11 of β-strands) of module 5 of repebody r-D3 were subjected to random variation.

Repebodies r-D3E5, r-D3E8 and r-D3E10 having a high binding affinity for hIL-6 were selected in the same manner as that in Examples 2-2 and 2-3, and among them, repebody r-D3E8 (SEQ ID NO: 8) having the highest binding affinity of 2.5 nM for hIL-6 was selected, suggesting that repebody r-D3E8 exhibits a 20-fold higher binding affinity than that of repebody r-D3 (see Table 2 and FIG. 2(c)).

TABLE 2
Substituted residues

| | Module 2 | | | | Module 3 | | | Module 4 | | | Module 5 | | | | K_{D} | IC₅₀ |
|---|---|---|---|---|---|---|---|---|---|---|---|---|---|---|---|---|
| Clone | 102 | 104 | 106 | 107 | 126 | 128 | 129 | 150 | 152 | 153 | 172 | 174 | 176 | 177 | (nM) | (nM) |

First round (library module 3, 4)

| | | | | | | | | | | | | | | | | |
|---|---|---|---|---|---|---|---|---|---|---|---|---|---|---|---|---|
| r-D3 | Y | A | G | G | T | E | P | Q | W | A | Y | N | A | H | 47 ± 3 | 1.5 |

Second round (library module 5)

| | | | | |
|---|---|---|---|---|
| r-D3E8 | | T | 2.5 ± 8.5 | 1.32 |
| r-D3E5 | M | F | 9.0 ± 0.9 | |
| r-D3E10 | | W | 13 ± 1.7 | |

Third round (library module 2)

| | | | | | | |
|---|---|---|---|---|---|---|
| r-D3E8C4 | K | T | V | S | 0.40 ± 0.08 | 0.51 |
| r-D3E8B2 | | T | V | Q | 1.18 ± 0.11 | |
| r-D3E8B3 | | T | Q | S | 0.95 ± 0.13 | |
| r-D3E8C9 | | | S | S | 0.54 ± 0.12 | |
| r-D3E8H2 | | L | R | S | 1.04 ± 0.09 | |

This means that the binding affinity of r-D3 for hIL-6 can be remarkably increased only by single variation of H177F in r-D3.

### Example 2-6

### Construction of Tertiary Library for Module Evolution

For the purpose of the additional improvement of repebody r-D3E8 selected in Example 2-5, a tertiary library was constructed by the module-by-module method. The construction of the tertiary library was performed in the same manner as that in Example 2-1, and four positions of module 1 of repebody r-D3E8 were subjected to random variation.

Repebodies r-D3E8C4, r-D3E8B2, r-D3E8B3, r-D3E8C9 and r-D3E8H2 having a high binding affinity for hIL-6 were selected in the same manner as that in Examples 2-2 and 2-3, and among them, repebody r-D3E8C4 (SEQ ID NO: 9) having the highest binding affinity of 397 nM for hIL-6 was selected, suggesting that repebody r-D3E8C4 exhibits a 118-fold higher binding affinity than that of repebody r-D3 (see Table 2 and FIG. 2(d)).

### Example 3

### Carrying Out of Reasonable Design for Increasing the Binding Affinity of Repebody Based on Complex Structure

Repebody-D3E8 (SEQ ID NO: 8) obtained in Example 2 was used as a polypeptide for a reasonable design. The polypeptide repebody-D3E8 binds to IL-6 with an affinity corresponding to a dissociation constant of 2.5 nM.

### Example 3-1

### Construction of Repebody/IL-6 Complex Structure

For a reasonable design, repebody-D3E8 and IL-6 were expressed in E. coli. The polypeptide repebody-D3E8 was purified using an Ni-NTA column and gel permeation chromatography (GPC), and then the complex was reacted in crystallization buffer (0.1M magnesium formate, 15-18% PEG3350) at a total concentration of 60 mg/ml at 17° C., thereby obtaining a crystalline structure. The structure of the complex was observed by an X-ray diffraction method (see FIG. 4, resolution: 2.3 Å).

### Example 3-2

### Analysis of Interaction Between Proteins Based on Complex Structure

Based on the complex structure obtained in Example 3-1, each residue in the repebody was analyzed. As a result, it could be seen that electrostatic interaction is a major factor related to binding affinity. It was judged that the optimization of electrostatic interaction can lead to an increase in the interaction between proteins. Based on this judgment, the present inventors analyzed the interaction type of repebody residue positioned adjacent to IL-6 in the complex structure.

### Example 3-3

### Reasonable Design for Increasing the Binding Affinity of Repebody, Based on the Results of Structural Analysis

Based on the results of analysis performed in Example 3-2, the present inventors performed a process for optimizing the electrostatic interaction between repebody residues. It was found that, among the amino acids of repebody close to IL-6, isoleucine at position 82 and asparagine at position 84 were positioned close to positively charged glutamic acid. Thus, each of the amino acids at positions 82 and 84 was substituted with positively charged lysine. Changes in the affinities of the mutations for IL-6 were observed by isothermal titration calorimetry (ITC) (Table 3). Because threonine at position 126 was adjacent to a hydrophobic site comprised of tryptophan at position 152, it was substituted with valine in order to induce further enhanced hydrophobic binding. Also, arginine at position 222 and tyrosine at position 244 were positioned close to positively charged arginine and lysine, respectively, and thus these residues were substituted with negatively charged glutamic acid having a relatively long length. Based on a reasonable design method that optimizes electrostatic interaction based on the results of the above-described structural analysis, it was found that all repebodies excluding asparagine at position 84 had a dissociation constant of about 50-9300 μM. In other words, the repebodies had an increased binding affinity for IL-6. Among them, repebody-D3E8-KE (SEQ ID NO: 10) had a dissociation constant of 63±14 μM, suggesting that it can effectively bind to interleukin-6.

TABLE 3

| | | Interaction | | Fold | IC₅₀ |
|---|---|---|---|---|---|
| Repebody | Mutation | Type | K_{D} (pM) | increase | (nM) |
| | Rb-D3E8 | | 2470 ± 45 | 1.0 | 1323 |
| D3E8 (I82K) | I82K | Ionic | 117 ± 51 | 21 | 34.3 |
| D3E8 (N84K) | N84K | Ionic | 9259 ± 1714 | 0.27 | |
| D3E8 (T126V) | T126V | Hydro- | 240 ± 153 | 10 | |
| | | phobic | | | |
| D3E8 (R222E) | R222E | Ionic | 214 ± 26 | 12 | |
| D3E8 (Y244E) | Y244E | Ionic | 236 ± 66 | 10 | |
| D3E8 (I82K, | I82K, | | 2500 ± 358 | 1.0 | |
| T126V) | T126V | | | | |
| D3E8-KE | I82K, | | 63 ± 14 | 39 | 2.1 |
| (D3E8 (I82K, | R222E | | | | |
| R222E) | | | | | |

### Example 4

### Treatment of Non-Small-Cell Lung Cancer Cells with Polypeptide

In order to evaluate the biological activities of the repebodies having increased binding affinities, the inhibitory effects of the repebodies on the activity of IL-6 were examined.

### Example 4-1

### Culture of Non-Small-Cell Lung Cancer Cells

First, a human non-small-cell lung cancer (H1650) cell line was suspended in medium [RPMI (Gibco-BRL, Grand Island, N.Y., USA) with 10% fetal bovine serum (Gibco-BRL), sodium pyruvate, nonessential amino acids, penicillin G (100 IU/ml) and streptomycin (100 mg/ml)] at a concentration of 1×10⁵ cells/ml and cultured in a 100-pi cell culture dish under the conditions of 37° C. and 5% CO₂.

### Example 4-2

### Treatment with Polypeptide (Repebody)

To the non-small-cell lung cancer cell medium prepared in Example 4-1, repebody-D3E8 (182K) (SEQ ID NO: 11) and repebody-D3E8C4 (SEQ ID NO: 9) that are selected in Example 2, and repebody-D3E8-KE (SEQ ID NO: 10), repebody-D3E8 (SEQ ID NO: 8) and repebody-D3 (SEQ ID NO: 7) that are selected in Example 3 were added at concentrations of 0.1, 1 and 10 mg/ml. As controls, an anti-interleukin-6 monoclonal antibody and an isotype control were added at a concentration of 1 mg/ml, and the cell medium was incubated for 18 hours.

### Example 4-3

### Analysis of Effects of Repebody on STAT3 and Interleukin-6

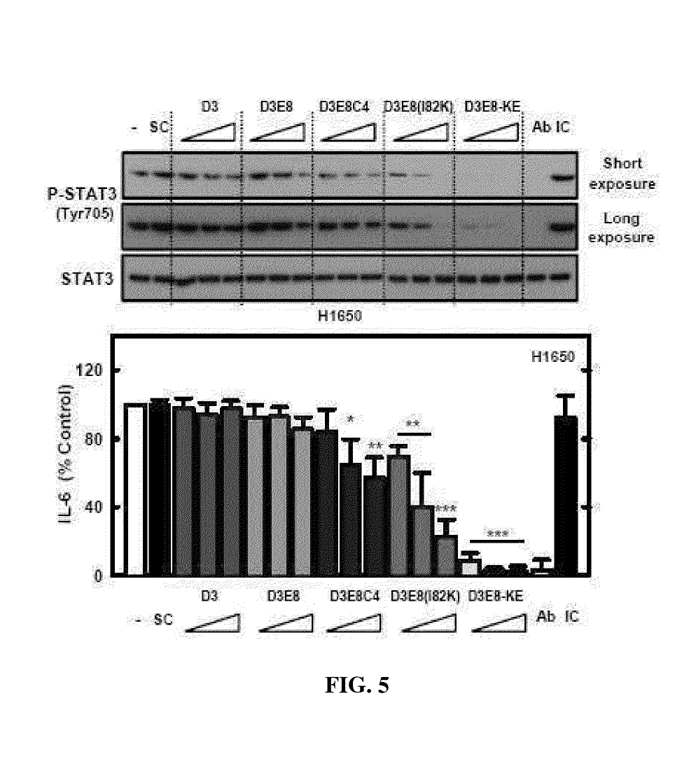

The medium of the non-small-cell lung cancer cells treated with the repebody in Example 4-2 were collected, and an enzyme immunoassay for interleukin-6 in the collected medium was performed. The cells were collected, and Western blot analysis for STAT3 in the collected cells was performed. The results of the analysis are shown in FIG. 5.

As can be seen in FIG. 5, the intracellular STAT3 activity (P-STAT3) and the production of interleukin-6 were significantly decreased by treatment with the repebody in a concentration-dependent manner. Particularly, it could be seen that D3E8 (182K) (SEQ ID NO: 11) and D3E8-KE (SEQ ID NO: 10), selected in the present invention, had excellent effects on the inhibition of intracellular STAT3 activity and interleukin-6 production compared to other repebodies (D3 and D3E8), and among them, D3E8-KE (SEQ ID NO: 10) showed inhibitory abilities similar to those of the control anti-interleukin-6 monoclonal antibody.

### Example 5

### Analysis of Effect of Repebody on Cell Viability

For the non-small-cell lung cancer cells treated with each of the polypeptides and the controls at a concentration of 1 mg/ml in Example 4-2, an MTT assay was performed to determine the viability of the cells. The medium of the non-small-cell lung cancer cells treated with each of the polypeptides and the controls at 1-day intervals for a total of 4 days was removed, and then the cells were incubated with an MTT tetrazolium solution for 4 hours. The solution was removed, and then DMSO was added and reacted with the cells for 15-20 minutes, after which the absorbance at a wavelength of 540 nm was measured using an ELISA reader. The results of the measurement are shown in FIG. 6.

As a result, it could be seen that the viability of the cells was lower in the order of D3, D3E8, D3E8C4, D3E8 (182K) and D3E8-KE. Among them, repebody D3E8-KE (SEQ ID NO: 10) showed a cell killing ability similar to that of the control anti-interleukin-6 monoclonal antibody. In other words, from the results in FIGS. 5 and 6, it could be seen that the cell death of the non-small-cell lung cancer cells was induced by treatment with the polypeptides of the present invention.

### Example 6

### Analysis of Effect of Repebody on Xenograft Mouse Model

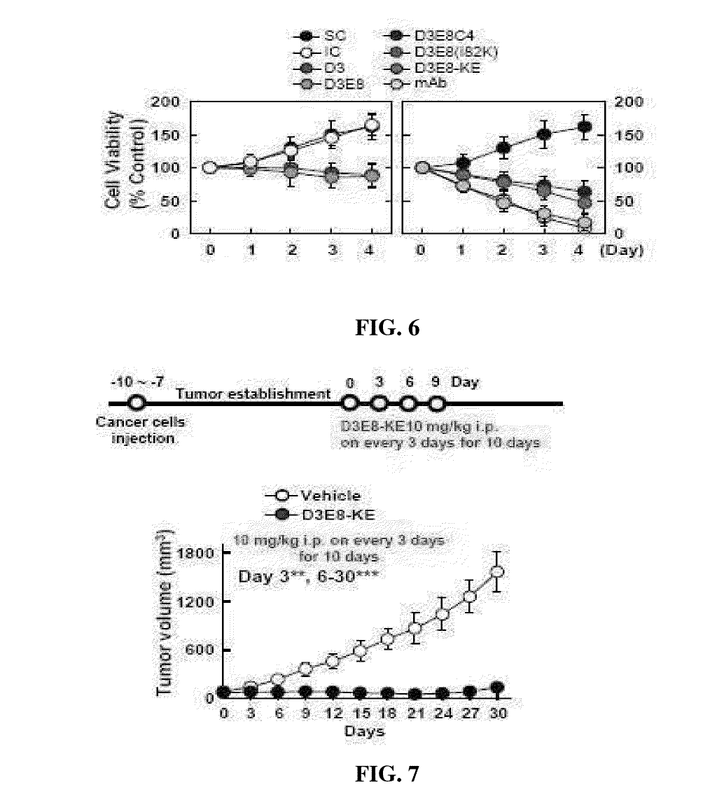

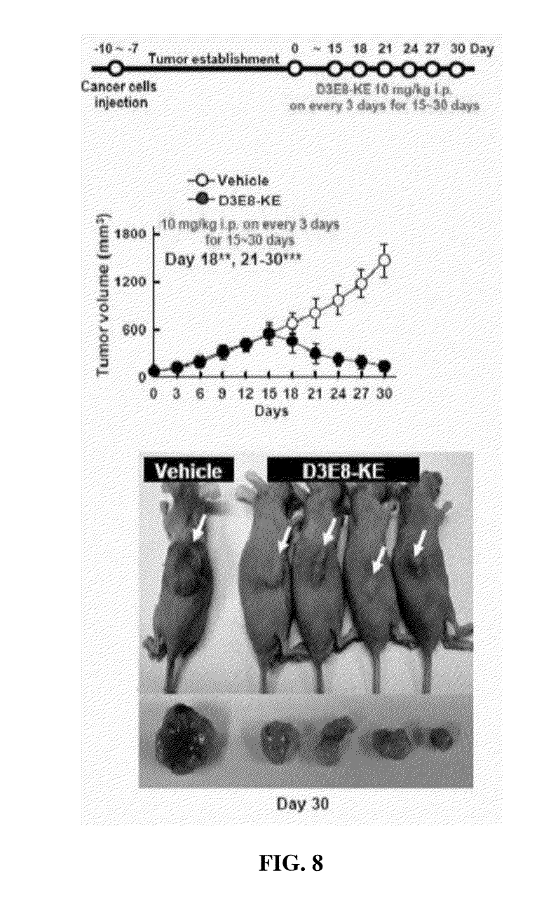

5×10⁶ non-small-cell lung cancer cells were injected subcutaneously into the right side of nude mice to construct xenograft mouse models. Then, repebody D3E8-KE (SEQ ID NO: 10) was injected intraperitoneally into the mouse models at a dose of 10 mg/kg, four times at 3-day intervals for 10 days. As a control, PBS was used. The volume of the tumor was measured at 3-day intervals and calculated according to the following equation: tumor volume=(length×width)/2. The results of the calculation are shown in FIG. 7. As a result, it could be seen that the volume of the tumor in the group treated with D3E8-KE (SEQ ID NO: 10) significantly decreased.

Meanwhile, xenograft mouse models injected with non-small-cell lung cancer cells were treated with the repebody, and the tumor inhibitory activity of the repebody was analyzed to evaluate the effect of the polypeptide on tumors. Specifically, tumors were allowed to grow actively for 15 days, and then D3E8-KE was injected intraperitoneally at a concentration of 10 mg/kg, five times at 3-day intervals for 15 days. As a control, PBS was used. The tumor volume was measured at 3-day intervals, and the results of the measurement are shown in FIG. 8. As can be seen in FIG. 8, the volume of the actively growing tumor was significantly decreased by treatment with D3E8-KE.

### INDUSTRIAL APPLICABILITY

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A repebody protein library comprising two or more repebodies having the ability to bind to a target, in which each of the repebodies is composed of repeat modules, each consisting of two or more amino acids, in which each repebody has a concave region serving to recognize a biomolecule and a convex region serving to maintain the structure thereof, the concave region and the convex region being formed by the repeat modules, and in which at least one amino acid in the repeat modules of the repebodies is different between the repeat modules.

2. The repebody protein library of claim 1, wherein the repeat modules are 2-30 in number.

3. The repebody protein library of claim 1, wherein the repeat modules are directly linked to each other.

4. The repebody protein library of claim 2, wherein the repeat modules are linked together by a (poly)peptide linker.

5. The repebody protein library of claim 1, wherein a protein, from which the N-terminus or C-terminus of the repeat modules is derived, is different between the repeat modules.

6. The repebody protein library of claim 1, wherein the N-terminus of the repeat modules is the N-terminus of an LRR (leucine rich repeat) protein that is of microbial origin and has an alpha helical capping motif.

7. The repebody protein library of claim 6, wherein the N-terminus of the protein is a fusion polypeptide, which has an amino acid sequence of SEQ ID NO: 12 and is composed of the following repeat module pattern:
LxxLxxLxLxxN
wherein L is alanine, glycine, phenylalanine, tyrosine, leucine, isoleucine, valine or tryptophan, N is asparagine, glutamine, serine, cysteine or threonine, and x is a hydrophilic amino acid.

8. The repebody protein library of claim 7, wherein the protein is selected from the group consisting of internalin protein A, internalin protein B, internalin protein C, internalin protein H and internalin protein J.

9. The repebody protein library of claim 5, wherein the C-terminus of the repeat modules is of microbial origin and is the C-terminus of VLR (Variable Lymphocyte Receptor) protein.

10. The repebody protein library of claim 1, wherein the repeat modules are modified repeat modules of VLR (Variable Lymphocyte Receptor) protein.

11. The repebody protein library of claim 10, wherein the modified repeat modules of the VLR protein comprise the following repeat module pattern:
LxxLxxLxLxxN
wherein L is alanine, glycine, phenylalanine, tyrosine, leucine, isoleucine, valine or tryptophan, N is asparagine, glutamine, serine, cysteine or threonine, and x is any amino acid.

12. A nucleotide library encoding the repebody protein library of claim 1.

13. A method for constructing the nucleotide library of claim 12, the method comprising the steps of:
(a) confirming repeat modules in a template repebody, and determining amino acid residues that are to be used to randomize a selected region of the repeat modules, in view of interaction with a target substance; and
(b) constructing a combination of nucleotide sequences encoding one or more of the repeat modules determined in step (a).

14. A method for improving a repebody protein, the method comprising the steps of:
(a) confirming a concave region of a template repebody, which serves to recognize a biomolecule, and a convex region that serves to maintain the structure of the template repebody, and determining amino acid residues to be randomized, in view of interaction with a target substance;
(b) constructing a protein library including repeat modules having the randomized amino acid residues;
(c) selecting, from the protein library, a repebody having an increased binding affinity for the target substance;
(d) confirming a mutated region in the repebody selected in step (c), and determining amino acid residues to be randomized in modules adjacent to the mutated region;
(e) constructing a protein library including repeat modules having the randomized amino acid residues determined in step (d);
(f) selecting, from the protein library of step (e), a repebody having an increased binding affinity for the target substance; and
(g) repeating steps (d) to (f) n times.

15. The method of claim 14, wherein the library in step (b) is constructed by a phage display method.

16. The method of claim 15, wherein the phage display method is a method of phage-displaying a product obtained from a template repebody-encoding DNA by an overlapping PCR method.

17. The method of claim 14, further comprising a step of performing additional mutation in a region of the selected repebody, which is to bind to the target substance, based on the structural analysis of a complex consisting of the selected repebody bound to the target substance.

18. The method of claim 14, wherein the protein library is a repebody protein library set forth in any one of claim 1.
